(19) United States
(12) Patent Application Publication
(10) Pub. No.: US 2023/0181728 A1
(43) Pub. Date: Jun. 15, 2023

(54) ADJUVANTED CONJUGATE OPIOID VACCINE

(71) Applicants: University of Houston System, Houston, TX (US); Tulane University, New Orleans, LA (US)

(72) Inventors: Colin N. Haile, Houston, TX (US); Gregory Cuny, Houston, TX (US); Elizabeth B. Norton, New Orleans, LA (US); Therese Kosten, Houston, TX (US)

(73) Assignees: University of Houston System, Houston, TX (US); Tulane University, New Orleans, LA (US)

(21) Appl. No.: 17/925,082

(22) PCT Filed: May 25, 2021
(86) PCT No.: PCT/US21/33961

§ 371 Date: Nov. 14, 2022

Related U.S. Application Data
(60) Provisional application No. 63/030,609, filed on May 27, 2020.

(51) Int. Cl. A61K 39/385; A61K 39/00; A61K 39/39; A61K 47/42; A61K 47/64; A61P 25/36; A61P 37/04

(52) U.S. Cl. CPC A61K 39/385; A61K 39/0013; A61K 39/39; A61K 47/42; A61K 47/6415; A61K 47/646; A61P 25/36; A61P 37/04; A61K 2039/6037; A61K 2039/55544; A61K 2039/575; A61K 2039/541; A61K 2039/543

(57) ABSTRACT

The adjuvanted conjugate opioid vaccine described herein is a conjugate of a protein carrier and at least one opioid backbone component or hapten conjugated thereto, admixed with at least one adjuvant. Anti-opioid effects are demonstrated after administration of a vaccine made up of the CRM197 protein carrier linked to a FEN backbone, combined with adjuvants such as dmLT or LTA1.

### BACKGROUND

This application claims priority to U.S. Provisional Patent Application No. 63/030,609, entitled “Adjuvanted Conjugate Opioid Vaccine,” filed May 27, 2020, the entire contents of which are hereby incorporated by reference.

This invention was made with government support under grant W81XWH-18-2-0044 awarded by the Department of Defense Alcohol and Substance Abuse Disorders Research Program and grant RO1A1114697 awarded by the National Institute of Allergy and Infectious Diseases of the National Institutes of Health. The government has certain rights in the invention.

This disclosure pertains to a conjugate opioid vaccine combined with various adjuvants.

Fentanyl and derivatives are highly addictive synthetic opioids that are also associated with fatal outcomes. Fatal outcomes result from accidental overdose and unintentional drug exposure. The prevalence of opioid used disorder and overdose deaths secondary to synthetic opioids continues to be a significant problem worldwide.

Although there are presently approved treatments for opioid use disorder (OUD), the pernicious epidemic in the United States and abroad suggests novel treatments are urgently needed. Recent statistics for the United States indicate an alarming increase in overdose deaths (72,306) in 2017 with nearly 50,000 of those deaths involving opioids. Of those deaths related to opioids, nearly 30,000 were linked to synthetic opioids. The distressing increase in overdose deaths among individuals with OUD is in part due to increased availability and use of fentanyl (FEN), a potent synthetic opioid. FEN is a mu opioid receptor agonist indicated for the treatment of moderate to severe pain. FEN and FEN-analogs (e.g. carfentanil, sufentanil, alfentanil, lofentanil) are highly lipophilic and rapidly penetrate the CNS (central nervous system) which is often lethal. The standard medication to reverse FEN's effects is the mu antagonist naloxone. Because of FEN's potency however, reversing an overdose requires very high and quickly administered doses of naloxone. Further, replacement therapies for OUD have considerable abuse liability themselves and marginally decrease relapse. An alternative treatment strategy that could potentially avert overdose and prevent relapse in individuals with OUD is vaccination with an anti-FEN vaccine.

Vaccines against drug targets show promise since they can induce the production of drug-specific antibodies and neutralize its biochemical activity. These antibodies bind to the drug sequestering it in the periphery preventing CNS penetration associated with euphoria or potential death by way of respiratory depression. Vaccines against substances associated with use disorders have been under development for decades. For example, vaccines against nicotine and cocaine were tested in Phase III clinical trials, but with limited success. The failure of previous vaccines was primarily due to low proportions of vaccinated subjects attaining sufficient antibody levels needed to bind to the drug and prevent CNS entry. Further, studies showed a majority of subjects failed to maintain high enough antibody concentrations in the periphery over time. Another factor to overcome as identified in past studies is high inter-subject variability in vaccine response. To surmount these factors an anti-FEN vaccine must produce high and enduring immunogenicity to the point at which low responders will have sufficient concentrations of anti-FEN for clinical efficacy.

### SUMMARY

The present disclosure relates generally to an anti-opioid vaccine. When admixed with various adjuvants, the vaccine generates substantive anti-opioid antibodies that block the opioid's analgesic effects and lethality.

Generally, the adjuvanted conjugate opioid vaccine disclosed herein includes a protein carrier (CRM197) linked to a fentanyl (FEN) backbone, combined with selected adjuvants. Administration of embodiments of the vaccine admixed with adjuvants resulted in high and enduring anti-opioid antibody levels. This effect corresponded with increased antibody secreting cells in bone marrow. Compared to unvaccinated rats, rats that received the adjuvanted conjugate vaccine showed comprehensive blockade of fentanyl's analgesic effects and complete protection from the lethal effects of the drug. Overall, embodiments of the vaccine may address the pressing need for an efficacious treatment for opioid use disorder and associated adverse events.

### BRIEF DESCRIPTION OF THE DRAWINGS

FIG. 1 shows a synthesis scheme for a preferred embodiment of an adjuvanted conjugate opioid vaccine.

FIG. 2 shows (A) brightfield and chemiluminescence overlay images of SDS-PAGE/Western blot in comparison with FEN-BSA control. The blot was first subjected to Ponceau staining to identify all proteins in the FEN-CRM antigen preps. The blot was then stripped and probed for the FEN hapten (a-PEN) (Lane 4). The blot was again stripped and re-probed for the carrier protein (a-CRM) confirming conjugation. Results were compared to and (B) anti-fentanyl serum IgG antibody levels in mice analyzed by ELISA 7 days post-boost with raw data shown in the top and analyzed data in the bottom.

FIG. 3 shows (A) serum dilutions of anti-FEN antibody concentration between groups vaccinated with FEN-CRM admixed with either Alum, dmLT, or LTA 1 at 0 and 3 weeks and (B) actual serum concentrations.

FIG. 4 shows (A) anti-FEN ELISPOT IgG/IgA plate showing analysis of mice bone marrow cells and (B) anti-FEN IgG ASC/1e6 (BM) concentrations at 2 and 6 weeks post boost.

FIG. 5 shows (A) anti-FEN IgG levels and (B) anti-PEN ASCs after sublingual (SL) or intranasal (IN) boost.

FIG. 6 shows results of tail flick assay in mice following dosage with fentanyl.

FIG. 7 shows serum anti-fentanyl antibody levels in male rats at 4, 6, and 8 weeks after vaccination.

FIG. 8 shows results of tail flick assay in vaccinated and non-vaccinated male rats after dosage with (A) 0.05 mg/kg and (B) 0.1 mg/kg fentanyl.

FIG. 9 shows results of hot plate assay in vaccinated and non-vaccinated male rats after dosage with (A) 0.05 mg/kg and (B) 0.1 mg/kg fentanyl.

FIG. 10 shows results of tail flick assay in vaccinated and non-vaccinated female rats after dosage with (A) 0.05 mg/kg and (B) 0.1 mg/kg fentanyl.

FIG. 11 shows results of hot plate assay in vaccinated and non-vaccinated female rats after dosage with (A) 0.05 mg/kg and (B) 0.1 mg/kg fentanyl.

FIG. 12 shows (A) lethality of high dose of fentanyl in non-vaccinated and vaccinated male rats and (B) anti-FEN antibody levels after fentanyl dosage at 6 months following initial vaccination in both male and female rats.

FIG. 13 shows brain and serum levels of fentanyl in vaccinated and non-vaccinated (A) male and (B) female rats following 0.1 mg/kg fentanyl.

### DETAILED DESCRIPTION OF PREFERRED EMBODIMENTS

The present disclosure relates to an anti-opioid vaccine. The vaccine is a conjugate and mixed with various adjuvants.

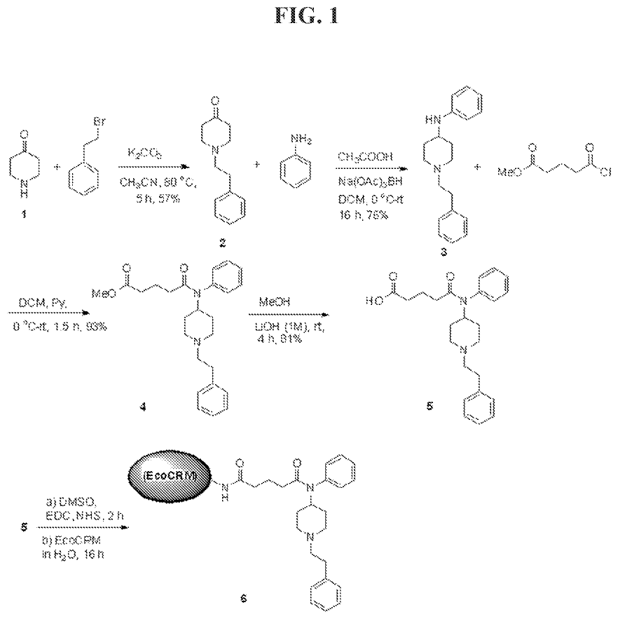

In preferred embodiments, the adjuvanted conjugate opioid vaccine described herein is a conjugate of a protein carrier and at least one opioid backbone component or hapten conjugated thereto. Ideally, the protein carrier should not produce an immune response. In additional preferred embodiments, the CRM197 protein carrier linked to a FEN backbone is the conjugate entity of the vaccine. CRM197 is a form of detoxified diphtheria toxin, which has a history of safety and efficacy in conjugate vaccines for adults and infants. Importantly CRM197 has demonstrated reduced adverse events with pre-existing immunity compared to other common carrier proteins (e.g., tetanus toxoid, diphtheria toxoid). FEN-CRM has about 12 FEN haptens linked to the CRM protein through surface lysine residues. A synthesis scheme for FEN-CRM illustrated is shown in FIG. 1. A covalent bond attaches a glutaryl linker to the anilide group in FEN (fentanyl-glutaryl) to form the FEN hapten. Construction of this attachment point to FEN is to prevent steric hindrance of the core N-(1-phenethylpiperidin-4-yl)-N-phenylacetamide scaffold that is shared by virtually all FEN derivatives. The terminal carboxylic acid on the glutaryl linker enabled 1-Ethyl-3-(3-dimethyl-aminopropyl) carbodiimide (EDC)/ N-hydroxysuccinimide (NHS) ester formation for bioconjugation of the hapten to the CRM carrier protein.

A unique component of the vaccine is the vaccine formulation itself whereby the conjugate, which may be FEN-CRM, is combined with adjuvants such as dmLT or LTA 1. The history and development of dmLT, or double mutant of heat-labile toxin (LT) from E. coli specifically, has been reviewed in detail. In short, dmLT was generated by introducing two mutations, R192G and L211A, into LT enterotoxin in order to divorce oral enterotoxicity from adjuvanticity. Notably, dmLT has been found safe by intramuscular, oral, sublingual, intradermal routes in humans. There has been extensive characterization of dmLT along with other documented improvement of parenteral and mucosal immunity to bacterial and viral antigens following oral, transcutaneous, sublingual, intradermal and intramuscular delivery with dmLT in animal models. dmLT promotes a mixed Th1/Th2/Th17 and IgA response similar to LT. This induction of Th17 cells is also a distinctive feature of dmLT, benefiting germinal center development which is key to generation of high titer antibodies.

dmLT cannot be used for respiratory delivery, as LT and older AB₅ mutants elicited Bell's palsy when delivered intranasal (IN) during 1990s clinical trials, including a short-lived commercial influenza vaccine. This is a direct consequence of B-subunit neuronal ganglioside binding combined with the inflammatory response to the vaccine. For this reason, an LTA1 adjuvant was developed that lacks the B-subunit and contains only a portion of dmLT's (and LT's) Al domain. Mice treated with up to 30pg LTA1 exhibit no signs of the intranasal toxicity that was seen with enterotoxin treatment, including inflammation in the olfactory epithelium or alterations in nerve function through olfactory testing. Like dmLT, LTA1 also activates antigen presenting cells and improves immune responses to co-delivered antigens. LTA1 improves antibody and cellular vaccine response to bacterial antigens by intranasal or intradermal delivery. LTA1 also improves antibody responses to viral antigens, including studies with intranasal flu vaccination where both survival and weight loss after influenza challenge in mice was improved with LTA1 adjuvant inclusion. Importantly, the adjuvants dmLT and LTA1 have never been reported for efficacy in substance abuse vaccines prior to these studies.

Accordingly, preferred embodiments disclosed herein relate to an adjuvanted conjugate opioid vaccine comprising a protein carrier, at least one opioid backbone hapten conjugated to the protein carrier, and at least one adjuvant admixed with the protein-hapten conjugate. In preferred embodiments of the opioid vaccine, the protein carrier is CRM197 and the opioid backbone hapten is fentanyl, where the fentanyl hapten is covalently attached to CRM197 through a glutaryl linker. In additional preferred embodiments of the opioid vaccine, the protein carrier is conjugated to at least one fentanyl hapten and no more than twenty fentanyl haptens.

In additional preferred embodiments, the adjuvanted conjugate opioid vaccine comprises a protein-hapten conjugate having the following structure:

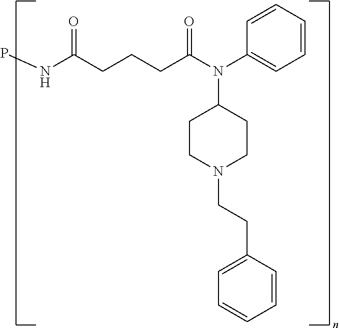

wherein P is a protein carrier, wherein P is preferably CRM197, wherein n is at least 1, and wherein n is preferably between 1 and 15.

In additional preferred embodiments of the adjuvanted conjugate opioid vaccine, the protein-hapten conjugate is admixed with one or more adjuvants. The adjuvants are preferably dmLT, LTA1, or combinations thereof. Other suitable adjuvants include alum (aluminum hydroxide, aluminum hydroxyphosphate sulfate), squalene based oil-in-water, MPLA-SM (TLR-4 agonist), flagellin derivatives (TLR-5 agonist) and saponin based adjuvants (TQL 1055, QS-21) among others.

The exemplary adjuvanted conjugate opioid vaccines described herein may occur in different geometric and enantiomeric forms, and both pure forms and mixtures of these separate isomers are included in the scope of this invention, as well as any physiologically functional or pharmacologically acceptable salt derivatives or prodrugs thereof. Production of these alternate forms would be well within the capabilities of one skilled in the art.

The current invention also pertains to methods of prevention or treatment of opioid use disorder, or otherwise minimizing or inhibiting the effects of opioids in a subject, including the step of administering an adjuvanted conjugate opioid vaccine in accordance with preferred embodiments disclosed herein. In preferred embodiments, the methods of prevention or treatment for opioid use disorder, or for minimizing or inhibiting the effects of opioids in a subject, include the step of administering a compound that is a conjugate of CRM197 and fentanyl haptens in accordance with preferred embodiments disclosed herein.

In another aspect of the present invention there is provided a pharmaceutical composition including a therapeutically effective amount of an adjuvanted conjugate opioid vaccine in accordance with preferred embodiments disclosed herein and a pharmaceutically acceptable excipient, adjuvant, carrier, buffer or stabilizer. A “therapeutically effective amount” is to be understood as an amount of an exemplary adjuvanted conjugate opioid vaccine that is sufficient to show inhibition of the effects of an opioid. The actual amount, rate and time-course of administration will depend on the opioid, the subject, the opioid dosage, and the nature and severity of the effects. Prescription of treatment is within the responsibility of general practitioners and other medical doctors. The pharmaceutically acceptable excipient, adjuvant, carrier, buffer or stabiliser should be non-toxic and should not interfere with the efficacy of the active ingredient. The precise nature of the carrier or other material will depend on the route of administration, which may be oral, sublingual, intranasal, or by injection, such as cutaneous, subcutaneous, or intravenous injection, or by dry powder inhaler.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such as gelatin. For intravenous, cutaneous or subcutaneous injection, the active ingredient will be in the form of a parenterally acceptable aqueous solution which is pyrogen-free and has a suitable pH, isotonicity and stability. Those of relevant skill in the art are well able to prepare suitable solutions using, for example, isotonic vehicles such as sodium chloride solution, Ringer's solution, or lactated Ringer's solution. Preservatives, stabilizers, buffers, antioxidants and/or other additives may be included as required.

In another aspect, there is provided the use in the manufacture of a medicament of a therapeutically effective amount of an adjuvanted conjugate opioid vaccine as defined above for administration to a subject.

The term “pharmacologically acceptable salt” used throughout the specification is to be taken as meaning any acid or base derived salt formed from hydrochloric, sulfuric, phosphoric, acetic, citric, oxalic, malonic, salicylic, malic, fumaric, succinic, ascorbic, maleic, methanesulfonic, isoethonic acids and the like, and potassium carbonate, sodium or potassium hydroxide, ammonia, triethylamine, triethanolamine and the like.

The term “prodrug” means a pharmacological substance that is administered in an inactive, or significantly less active, form. Once administered, the prodrug is metabolised in vivo into an active metabolite.

The term “therapeutically effective amount” means a nontoxic but sufficient amount of the drug to provide the desired therapeutic effect. The amount that is “effective” will vary from subject to subject, depending on the age and general condition of the individual, the particular concentration and composition being administered, and the like. Thus, it is not always possible to specify an exact effective amount. However, an appropriate effective amount in any individual case may be determined by one of ordinary skill in the art using routine experimentation. Furthermore, the effective amount is the concentration that is within a range sufficient to permit ready application of the formulation so as to deliver an amount of the drug that is within a therapeutically effective range.

Further aspects of the present invention will become apparent from the following description given by way of example only.

### EXAMPLE 1

A general scheme for synthesis of a CRM-FEN conjugate according to preferred embodiments disclosed herein is shown in FIG. 1 and described more fully below.

### N-Phenylethylpiperidin-4-one (2)

To a solution of 4-piperidone monohydrate hydrochloride 1 (2.0 g, 14.75 mmol) was dissolved in acetonitrile (40 mL) was added K2CO₃ (6.1 g, 44.14 mmol) and (2-bromoethyl)benzene 5 (2.46 g, 13.27 mmol) at ambient temperature. The resulting suspension was refluxed at 80° C. for 5 h. The reaction mixture was monitored by TLC (50% ethylacetate in hexane). Upon completion of the reaction, the reaction mixture was allowed to cool to room temperature and filtered through a small celite pad. The crude material was purified by flash column chromatography (0-30% ethyl acetate in hexane) to afford compound 2 (1.72 g, 57%) as off-white solid; mp 57-59° C. ¹H NMR (500 MHz, CDCl₃): δ 7.32-7.29 (m, 2H), 7.23-7.20 (m, 3H), 2.86-2.82 (m, 6H), 2.75-2.71(m, 2H), 2.49 (t, J=6.0 Hz, 4H); ¹³C NMR (125 MHz, CDCl₃): δ 209.1, 139.9, 128.6, 128.4, 126.2, 59.3, 53.0, 41.2, 34.1.

### 1-Phenethyl-N-phenylpiperidin-4-amine (3)

To a solution of aniline (90 μL, 0.98 mmol) in dichloromethane (3 mL) was added acetic acid (56 μL, 0.98 mmol) drop wise at 0° C. Subsequently N-phenylethylpiperidin-4-one 2 (200 mg, 0.984 mmol, dissolved in 1 mL dichloromethane) was added drop wise, followed by addition of sodium triacetoxyborohydride (313 mg, 1.476 mmol) in 3 portions over a 12 minutes timeframe. The reaction mixture was stirred at ambient temperature for 16 h. After completion of the reaction, methanol (3 mL) was added and the reaction mixture was partitioned between dichloromethane and sat. NaHCO₃. The organic phase was separated and washed with brine, dried over anhydrous Na₂SO₄ and filtered. Solvent was removed under reduced pressure providing the crude compound. This material was purified by flash column chromatography (0-50% ethylacetate in hexane) to obtain the desired compound 3 as a off-white solid (211 mg,76%). mp 96-98° C. ¹H NMR (500 MHz, CDCl₃): δ 7.31-7.28 (m, 2H), 7.22-7.15 (m, 5H), 6.68 (t, J=7.5 Hz, 1H), 6.60 (d, J=8.0 Hz, 2H), 3.52 (brs, 1H), 3.34-3.30 (m, 1H), 2.98-2.95 (m, 2H), 2.84-2.80 (m, 2H), 2.63-2.59 (m, 2H), 2.21 (t, J=10.9 Hz, 2H), 2.10-2.07 (m, 2H), 1.54-1.46 (m, 2H); ¹³C NMR (125 MHz, CDCl₃): δ 147.0, 140.3, 129.3, 128.7, 128.4, 126.0, 117.2, 113.2, 60.6, 52.4, 49.8, 33.8, 32.5.

### Methyl 5-oxo-5((1-phenethylpiperidin-4-yl)(phenyl)amino)pentanoate

To a solution of compound 3 (200 mg, 0.713 mmol) in anhydrous dichloromethane was added pyridine (112 mg, 1.415 mmol) and methyl 4-(chloroformyl)butyrate (129 mg, 0.783 mmol) at 0° C. The reaction mixture was stirred for 5-10 min at 0° C. The reaction mixture was allowed to warm to room temperature and then stir for 1.5 h. Reaction progress was monitored by TLC (5% methanol in dichloromethane). Upon completion, the reaction was quenched with sat. NaHCO₃ solution. The organic layer was separated and the aqueous layer was washed with ethyl acetate (2×10 mL). The combined organic layers were washed with brine, dried over hydrous Na₂SO_{4,} filtered and concentrated under reduced pressure providing the crude compound, which was purified by flash column chromatography (0-5% methanol in dichloromethane) to obtain the desired compound 4 as a pale yellow viscous liquid (273 mg, 93%). ¹H NMR (400 MHz, CDCl₃): δ 7.42-7.34 (m, 3H), 7.28-7.24 (m, 2H), 7.19-7.14 (m, 3H), 7.08-7.06 (m, 2H), 4.68 (tt, J=12.3 Hz, 3.9 Hz, 1H), 3.60 (s, 3H), 3.03-3.0 (m, 2H), 2.75-2.71(m, 2H), 2.56-2.52 (m, 2H), 2.27 (t, J=7.1 Hz, 2H), 2.20-2.14 (m, 2H), 1.98-1.94 (m, 2H), 1.91-1.79 (m, 4H), 1.49-1.39 (qd, J=12.4 Hz, 3.7 Hz, 2H); ¹³C NMR (100 MHz, CDCl₃): δ 173.6, 171.8, 140.0, 138.4, 130.3, 129.3, 128.6, 128.4, 126.0, 60.4, 53.0, 52.1, 51.4, 34.0, 33.7, 33.2, 30.4, 20.6.

### 5-Oxo-5-((1-phenethylpiperidin-4-yl)(phenyl)amino)pentanoic Acid

To a solution of ester 4 (100 mg, 0.244 mmol) in methanol was added 0.5 mL of 1M aqueous LiOH solution. The reaction mixture was stirred for 4 h at room temperature. The progress of the reaction was monitored by TLC (5% methanol in dichloromethane). Upon completion, the reaction solution was acidified with 3N aqueous HCl to pH 5. Then it was concentrated in vacuo to remove methanol. The residue was extracted with ethylacetate and washed with a small amount of brine solution (note, product is water soluble). The organic layer was dried over anhydrous Na₂SO₄, filtered and concentrated under reduced pressure. The crude compound was purified by flash column chromatography (0-5% methanol in dichloromethane) to yield the target compound 5 as an off-white solid (78 mg, 81%). mp 60-63° C.¹H NMR (400 MHz, CDCl₃): δ 8.93 (brs, 1H), 7.42-7.34 (m, 3H), 7.30-7.16 (m, 5H), 7.06-7.04 (m, 2H), 4.78-4.70 (m, 1H), 3.51-3.48 (m, 2H), 3.02 (s, 4H), 2.77-2.72 (m, 2H), 2.17 (t, J=7.1 Hz, 2H), 1.98 (t, J=7.3 Hz, 2H), 1.93-1.75 (m, 6H); ¹³C NMR (100 MHz, CDCl₃): δ 176.9, 172.6, 137.6, 136.7, 129.8, 129.7, 129.0, 128.8, 128.6, 127.0, 58.1, 51.9, 50.3, 33.9, 33.7, 30.7, 27.7, 20.6. HRMS (ESI): m/z [M+H]+ calcd for C₂₄H₃₁N₂O₃: 395.2329; found 395.2330.

Fentanyl-CRM Conjugation:

To a mixture of fentanyl acid 5 (0.6 mg, 2.0 equiv, 1.44×10^{—3} mmol), EDC (0.3 mg, 2.0 equiv, 1.44×10⁻³ mmol) and NHS (0.2 mg, 2.0 equiv, 1.44×10⁻³ mmol) in 315 μL DMSO was added. The reaction mixture was stirred at room temperature for 2 h. Then the resulting reaction mixture was added drop wise to a solution of CRM197 (EcoCRM™, Fina Biosolutions) (2.1 mg, 1 equiv, 7.19×10⁻⁴ mmol, dissolved in 2100 μL water) and then stirred overnight. Next, a dialysis column (Thermo Scientific) was prepared by adding approximately 44.5 mL of sterile 1×PBS into the bottom reservoir. The top reservoir was then rinsed with 500 μL of 1×PBS and placed into the bottom and reaction mixture introduced. Column was then sealed and placed onto an orbital shaker (180 rpm) for 2 hours. After agitation, the mixture was removed and placed into eppindorf tubes and volume recorded. The mixture is then loaded into a 5 mL syringe and sterilized by filtering it through a 0.2 μm HT Tuffryn membrane (Acrodisk syringe filter, Pall Corporation, Ann Arbor, Mich).

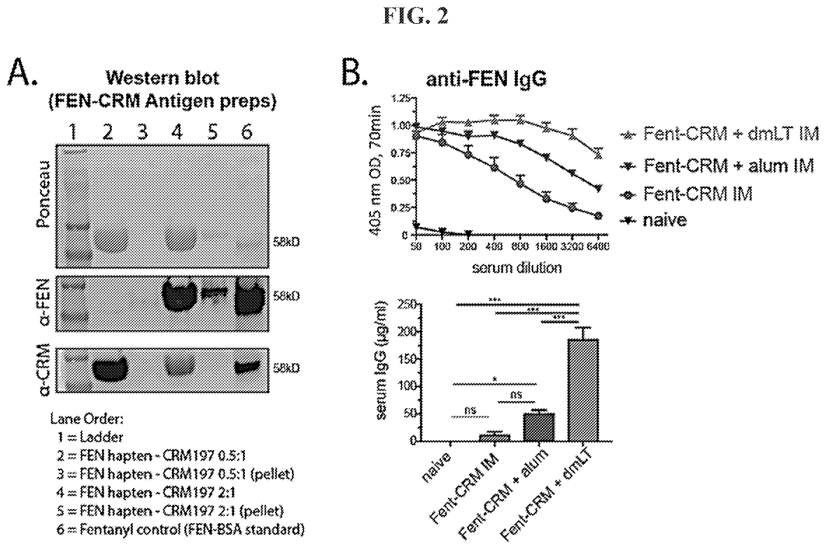

FEN hapten-CRM conjugates were prepared at different FEN hapten:CRM carrier ratios, i.e. 2 or 20 mols of FEN per mol CRM exposed lysine residues with the ratio of FEN hapten to coupling reagents remaining at 1:1. Test formulations were evaluated by SDS-PAGE/Western blot in comparison with FEN-BSA control by Ponceau staining (for all proteins), binding of anti-Fentanyl antibodies (a-FEN) or stripped and re-probed for anti-CRM197 antibodies (a-CRM) with brightfield and chemiluminescence overlay images shown in FIG. 2A. As shown in FIG. 2A, successful FEN-CRM conjugation through a distinct linkage was confirmed.

Subsequently, the 2:1 FEN-CRM formulation was dialyzed and used to vaccinate mice (Balb/c, N=5 per group, 1.5 μg/mouse) at 0 and 14 days. The formulation was intramuscularly injected in 20 μl alone in PBS buffer or with absorption 1:1 with 2% alyhydrogel (alum, 100 μg final done) or admixed with 1 μg dmLT.

An ELISA assay that detects anti-fentanyl antibodies at nanogram concentrations was developed and used to evaluate the exemplary vaccine. In brief, plates (Maxisorp, VWR) are first coated with BSA-1-EN (CalBioreagents, San Mateo, Calif.) for the capture antigen and mouse IgG (Sigma) for the standard curve in carbonate-bicarbonate buffer (Sigma) and incubated for one hour at 25° C. Following washing, plates were blocked with 2% skim milk in PBS-Tween20 (0.05%) for one hour. Then samples, positive (fentanyl monoclonal, Cal Bioreagents, San Mateo, Calif.) and negative (serum from unvaccinated animals) controls (diluted in assay buffer, 0.2% skim milk in PBS-T) were added directly to the appropriate wells and incubated for one hour. Plates were then washed and detection antibody (1:500) was added and plates were incubated for one hour. Plates underwent a final wash and 100 μL of substrate (1 mg/ml pNPP in diethanolamine, Sigma) was added to each well. Optical density (OD) for each well was obtained at 405 nm using a plate reader (Fischer Scientific). Antibody concentrations were then quantified using four parameter logistic curve fit for calculating symmetrical sigmoidal calibrators. Anti-fentanyl serum IgG antibody levels were analyzed by ELISA 7 days post-boost with raw data shown in the top of FIG. 2B and analyzed data shown in the bottom of FIG. 2B. Vaccination with FEN-CRM+dmLT resulted in significantly greater amounts of anti-fentanyl antibodies as determined with ELISA.

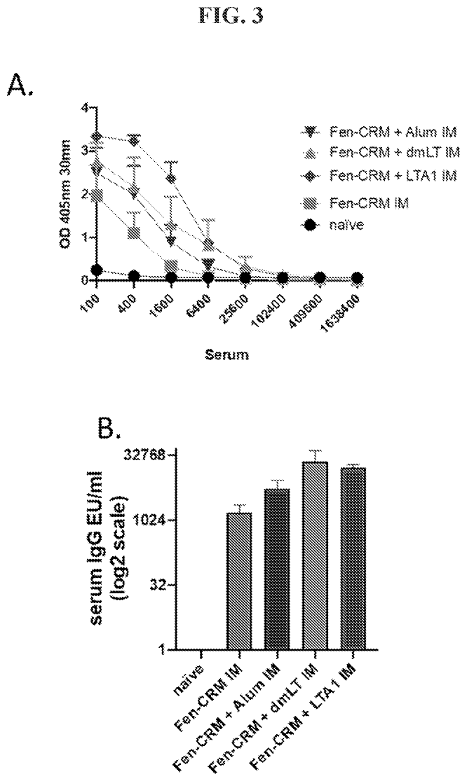

FIGS. 3A and 3B show that both dmLT and LTA1 increase anti-FEN IgG levels when combined with FEN-CRM. Mice (Balb/c, N=5/group) were vaccinated with FEN-CRM (5 μg) admixed with either Alum (150 dmLT (0.1 μg), or LTA 1 (5 μg) at 0 and 3 weeks. Serum anti-FEN antibody levels were determined with ELISA on week 5. FIG. 3A shows serum dilutions of anti-FEN antibody concentration between groups. FIG. 3B shows actual serum concentrations between groups (EU=ELISA Units).

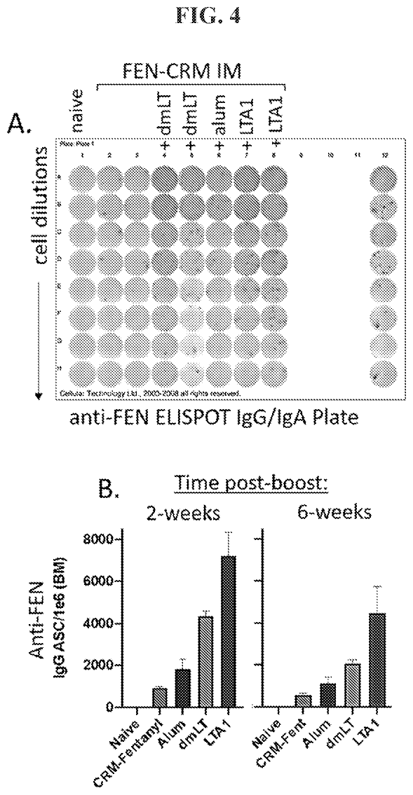

FIGS. 4A and 4B show that adjuvants dmLT and LTA1 induce high levels of FEN-specific antibody secreting cells (ASC) in the bone marrow. Mice (Balb/c, N=5/group) were vaccinated with PEN-CRM admixed with either Alum, dmLT or LTA1 in the same amounts as above at 0 and 3 weeks. Bone marrow cells were isolated and homogenized on day 35 and analyzed. IgG/IgA Antigen Secreting Cell (ASC) ELISPOTS were performed following the ImmunoSpot B-Cell Double-Color Enzymatic Mu B-Cell IgA/IgG ELISPOT kit (ImmunoSpot). Briefly, for ASCs, plates were coated with 0.1 μg/well of Tetanus Toxoid-Fentanyl conjugation antigen. Twenty-four hours later, splenocytes and bone marrow cells were plated in duplicate dilutions and 4 different concentrations starting from 1.0, 0.5, or 2.5e6 cells and incubated at 37° C. for 24 hours. Kit instructions were followed for detection, with overnight incubation of the Anti-Murine IgG/IgA detection solution and 20 min incubation with Blue and Red Developer Solutions. Plates were allowed to dry overnight and imaged with an ImmunoSpot S5 Macro Analyzer with ImmunoCapture 6.3.3. Spots were quantified with ImmunoSpot software. For memory B-cells analysis, cells were adjusted to 4e6 cells/ml in a 24-well non-tissue culture treated plate (Falcon) and incubated for 4 days at 37° C. in CTL-Test B culture medium with 1% L-Glutamine Pre-stimulated cells were harvested after 4 days, recounted, diluted and plated in the same manner as ASCs above. Spot counts were adjusted for dilution factor, and the most accurate dilution was average to yield antibody secreting cells per million. FIG. 4A shows an anti-FEN ELISPOT IgG/IgA plate. FIG. 4B shows anti-FEN IgG ASC/1e6 (BM) concentrations at 2 and 6 weeks post boost. Elevated anti-FEN antibodies relate to the ability of dmLT and LTA1 to stimulate the production of ACS in the bone marrow and this effect is enduring.

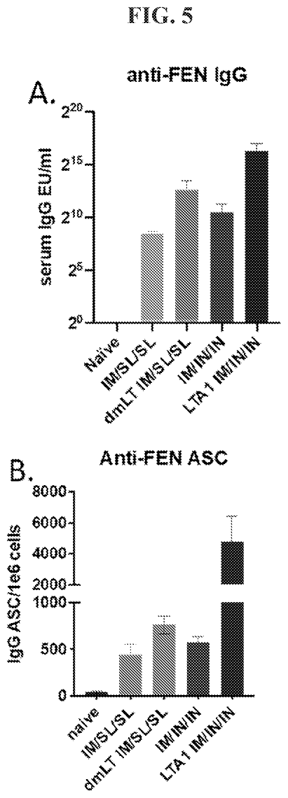

Further, as shown in FIGS. 5A and 5B, the ability of FEN-CRM combined with either dmLT or LTA1 to elevate anti-FEN antibodies and stimulate ASC is also achieved when the formulation is administered through mucosal routes, both sublingual (SL) and intranasal (IN). Mice were vaccinated in the same amounts as above (FEN-CRM (5 μg) admixed with either dmLT (0.1 μg) or LTA 1 (5 μg)). For both SL and IN vaccinations, mice were anesthetized using ketamine/zylazine and the vaccine was administered using a pipetman. Mice were supine and allowed to recover. Data was collected at week 8 after intramuscular (IM) vaccination at week 0 and SL or IN boost at weeks 3 and 6. anti-FEN IgG levels shown in FIG. 5A were determined using ELISA and the process described above. ASCs were harvested from bone marrow from left and right tibia and fibulas. Cells were filtered then counted (EU=ELISA units) with results shown in FIG. 5B.

Anti-nociceptive effects of FEN was assessed using a tail flick assay. For the tail flick test, radiant heat (25 IR) was applied to the tail 3 cm from the tip using a tail flick apparatus (Ugo Basile, Collegeville, Pa). The time from the onset of the heat to the withdrawal of the tail (latency) was recorded. To avoid tissue damage, the heat stimulus was discontinued after 10-s (cut-off latency). Baseline latencies to move the tail were determined three times (at least 1 minute inter-trial interval). Mice (Balb/c, N=5-20/group) were vaccinated at 0, 3 and 6 weeks with PBS (naïve), FEN-CRM (5 ₗig) alone, and FEN-CRM admixed with Alum or two different doses of dmLT. Latencies were determined at 10 minutes post-injection. Data generated from unvaccinated and vaccinated mice that received FEN-CRM admixed with adjuvants Alum and two doses of dmLT (0.1 and 1 μg) that underwent analgesic tests of fentanyl (30 and 100 ug/kg, IP) in the tail flick assay are presented in FIG. 6 (left to right—PBS (naïve), FEN-CRM (5 ₗig) alone, FEN-CRM admixed with alum, FEN-CRM admixed with dmLT (0.1 μg), and PEN-CRM admixed with dmLT (1 μg)). Overall, no significant differences were found at the low dose of FEN (30 μg/kg) whereas FEN-CRM +dmLT (1 μg) showed statistically significant blockade of FEN's analgesic effects at the high FEN dose (100 μg/kg). Data are presented as %MPE (percent Maximum Possible Effect−[(test latency-control latency)/(cutoff criterion-control latency)×100].

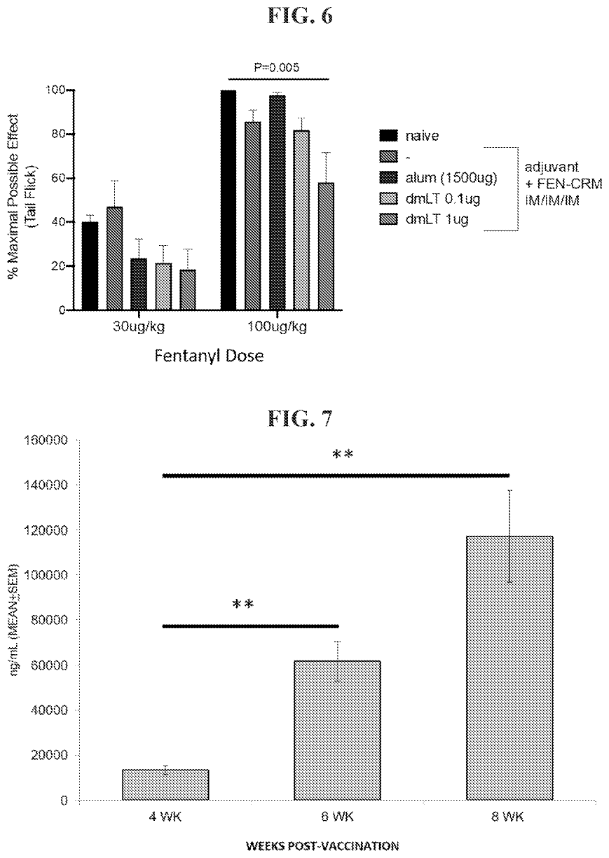

Additional nociception tests in male and female Sprague Dawley rats that received no vaccine or vaccinated with FEN-CRM (5 μg)+dmLT (1 μg) at 0, 3 and 6 weeks were conducted. Male (N=60) and female (N=60) Sprague Dawley rats were divided into groups (N=15/group) that received IM injections of either no vaccine (saline) or FEN-CRM (5 μg) conjugate admixed with adjuvant dmLT at 0, 3 and 6 weeks. Blood samples were taken at 4, 6 and 8 weeks and serum anti-FEN antibody concentrations determined by ELISA. Data from male rats (N=57-60/per time point) is presented in FIG. 7. Overall, anti-FEN antibody levels significantly increased over time (**P<0.0001), which was also the case for antibody levels in female rats (data not shown).

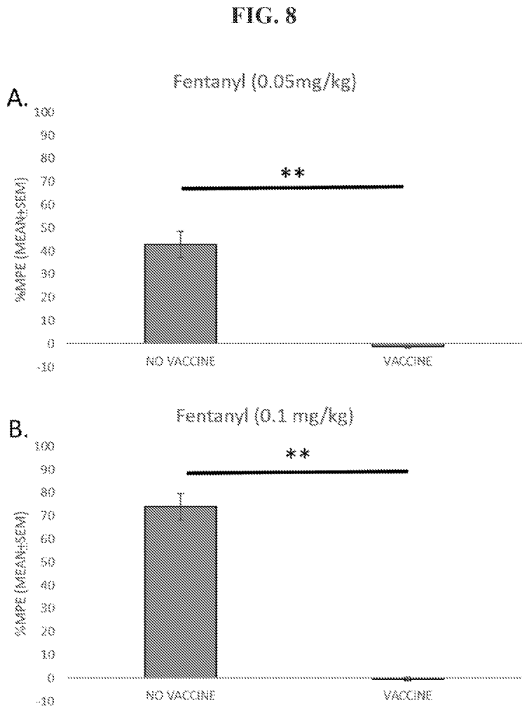

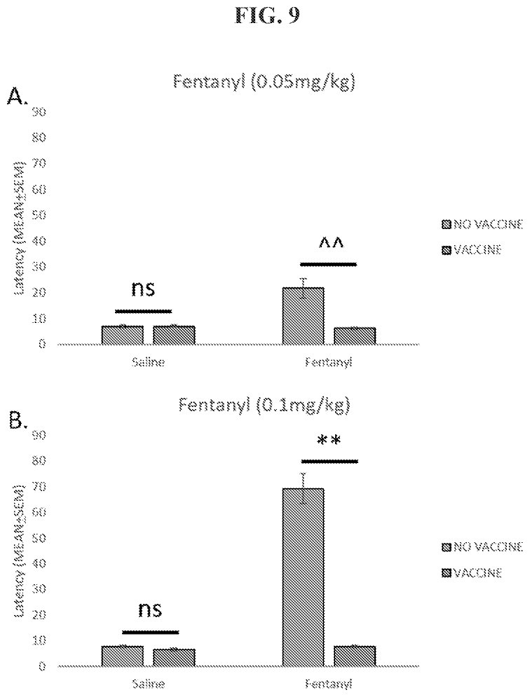

At approximately 14-15 weeks post-initial vaccination, nociception tests (tail flick and hot plate assays) were conducted following fentanyl dosing (0.05 and 0.1 mg/kg, SC). For the tail flick test, radiant heat (Ugo Basile, Collegeville, Pa.) was adjusted to produce an average tail flick latency of 2 seconds. During testing, heat was applied to the tail approximately 3 cm and the time from the onset of the heat to the withdrawal of the tail (latency) was recorded. Tail flick was assessed three times following saline (˜1 minute inter-trial interval). Rats were then administered one of two doses of fentanyl counterbalanced over days and tail flick again assessed. At least two days separated the two doses of fentanyl. For the hot plate test, rats were placed on a 53° C. hot plate (Hotplate Analgesia Meter, Columbus Instruments, Columbus, Ohio) and latency to first rear paw lick recorded. To avoid tissue damage, the cut-off time in which the animal was removed from the apparatus for this assay was 90 seconds. Baseline latencies were obtained following a SC injection of saline then one of two doses of FEN counterbalanced over days. As shown in FIGS. 8A and 8B, the vaccine completely blocked the analgesic effects of both doses of FEN (0.05 and 0.1 mg/kg) in all vaccinated male rats, whereas un-vaccinated rats showed robust analgesia. Data from the tail flick assay are presented as % MPE. Hot plate data are presented as mean (±SEM) latency (**P<0.00000001).

The complete blockade of FEN's analgesic effects in all rats was unexpected based on data obtained from studies using mice. FIGS. 9A and 9B present latency data from hot plate analgesic tests in this same group of male rats following the low (0.05 mg/kg) and high (0.1 mg/kg) doses of FEN. Rats (N=30/group) either received no vaccine or FEN-CRM conjugate vaccine (5 μg) with dmLT (1 μg) at 0, 3 and 6 weeks (IM). At approximately 14-16 weeks post-initial vaccination nociception tests (hot plate assays) were conducted following fentanyl dosing (0.05 and 0.1 mg/kg, SQ). Data from the hot plate assay are presented as latency to like the hind paw. Cut-off time was 90 seconds to prevent tissue damage (ns=no statistically significant difference, ^^P<0.001; **P<0.00000001). Consistent with results from the tail flick tests, FEN's analgesic effects were completely block in vaccinated rats. In contrast, potent FEN-induced analgesia was observed in rats that did not receive the vaccine.

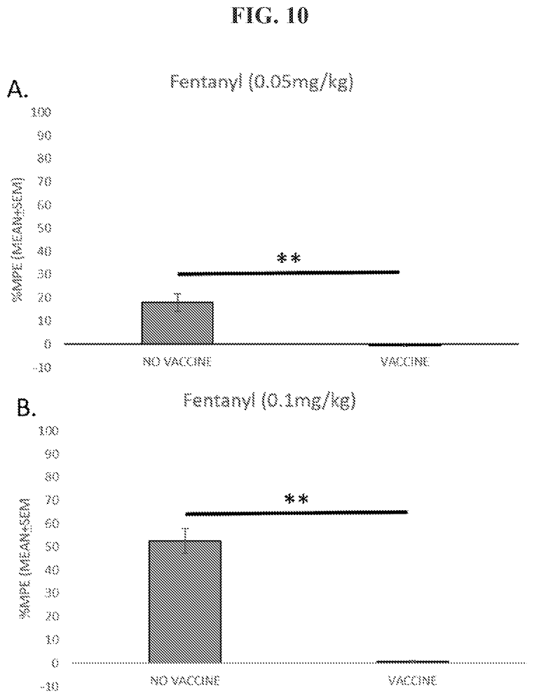

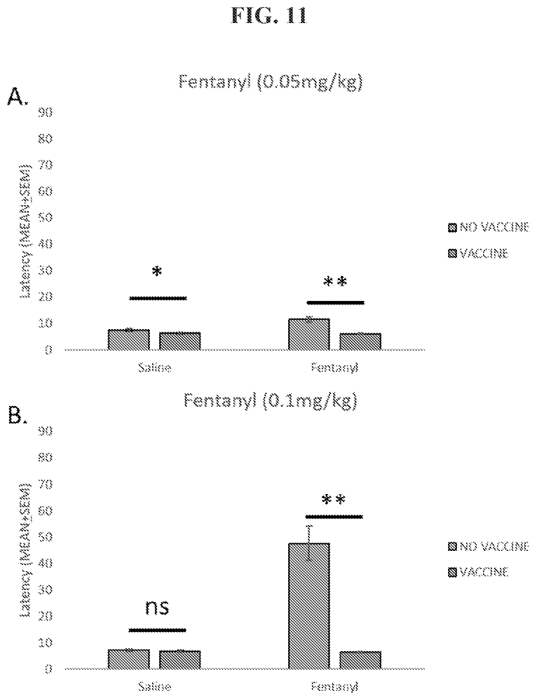

Tail flick assay data for female rats are presented in FIGS. 10A and 10B. Rats (N=29-30/group) either received no vaccine or FEN-CRM conjugate vaccine (5 μg) with dmLT (1 μg) at 0, 3 and 6 weeks (IM). At approximately 14-16 weeks post-initial vaccination nociception tests (tail flick) were conducted following fentanyl dosing (0.05 and 0.1 mg/kg, SQ). Data from the tail flick assay are presented as % MPE (**P<0.00000001). In accord with data from male rats, all vaccinated female rats showed complete blockade of the analgesic effects of both doses of FEN.

In addition, and also consistent with tests in male rats, increases in latency following fentanyl dosing were significantly blocked in all female vaccinated rats. Rats (N=29-30/group) either received no vaccine or FEN-CRM conjugate vaccine (5 μg) with dmLT (1 μg) at 0, 3 and 6 weeks (IM). At approximately 14-16 weeks post-initial vaccination nociception tests (hot plate assays) were conducted following fentanyl dosing (0.05 and 0.1 mg/kg, SC). Data from the hot plate assay are presented in FIGS. 11A and 11B as latency to lick the hind paw. Cut-off time was 90 seconds to prevent tissue damage (ns=no statistically significant difference, *P<0.05, **P<0.000001). This finding was also unexpected since anti-FEN antibody levels varied between vaccinated rats suggesting high affinity anti-FEN reserves.

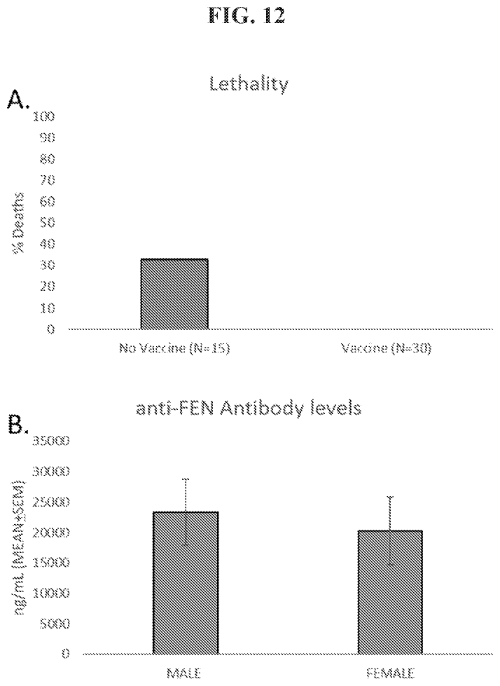

An additional potential use of the present vaccines is use as a protectant whether to prevent overdose or for individuals that are unintentionally exposed to potent synthetic opioids (e.g. first responders, police). Lethality data for male rats is presented in FIG. 12A. Rates either received no vaccine or FEN-CRM conjugate vaccine (5 μg) with dmLT (1 μg) at 0, 3 and 6 weeks (IM). One week after analgesic tests, all rats were administered an additional high dose of FEN (0.1 mg/kg) and then processed. Blood and brain samples were taken for analysis. Within 30 minutes, 33% of the rats that did not receive the vaccine unexpected perished. None of the vaccinated rats (Male=30, Female=30) perished. Serum anti-FEN antibodies at the time when the final FEN dose was administered are shown in FIG. 12B. Both male and female rats showed enduring anti-FEN antibody levels at 6 months post-initial vaccination. As noted, these levels were adequate to provide 100% protection against the high dose of FEN (0.1 mg/kg).

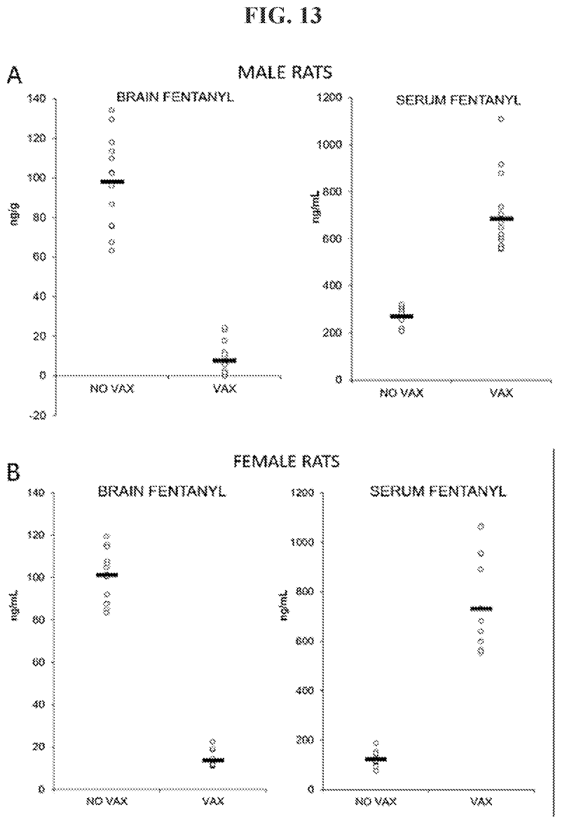

Results from an additional experiment are shown in FIG. 13, whereby vaccinated and non-vaccinated rats were administered fentanyl as noted above and brain and serum processed for fentanyl levels. Rats were administered 0.1 mg/kg (SC) fentanyl, then 30 minutes later they were anesthetized with isoflurane. A bilateral thoracotomy was performed and whole blood removed via left ventricle puncture with a 22-gauge needle then processed for serum. The rat was then perfused with cold 1× PBS (5 mL over 2 minutes) then the brain removed, and washed in 1×PBS and immediately placed on dry ice. Brain sample were stored at —80° C. until FEN levels were determined. Brain and serum samples were determined by a specialized ELISA (Guerrieri D, Kjellqvist F, Kronstrand R, Green H, Validation and Cross-Reactivity Data for Fentanyl Analogs with the Immunalysis Fentanyl ELISA, Journal of Toxicology, 2019; 43-24). The data indicates that fentanyl was sequestered in the periphery and prevented from entering the central nervous system in both male (FIG. 13A) and female (FIG. 13B) rats that received the vaccine. In contrast, fentanyl readily entered the brain in rats that did not receive the vaccine.

Overall, data presented herein supports the following conclusions:
1) Significant increases in serum anti-FEN antibodies were generated in mice following administration of PEN-CRM admixed with adjuvants dmLT or LTA1 compared to FEN-CRM alone and FEN-CRM+Alum.
2) Adjuvanted FEN-CRM with dmLT or LTA1 administered to mice produced greater amounts of antibody secreting cells (in bone marrow) compared to FEN-CRM alone and FEN-CRM+Alum.
3) Adjuvanted PEN-CRM with dmLT or LTA1 administered to mice via sublingual and intranasal respectively increased anti-FEN antibodies and generated greater amounts of antibody secreting cells (in bone marrow) compared to FEN-CRM alone.
3) The analgesic effects of FEN were significantly attenuated in mice vaccinated with FEN-CRM+dmLT.
4) Serum anti-FEN antibody levels in both male and female rats significantly increased over time following vaccination with FEN-CRM+dmLT.
5) Compared to un-vaccinated rats, complete blockade (100%) of FEN's analgesic effects was achieved in both male and female rats vaccinated with FEN-CRM+dmLT.

6) The FEN-CRM+dmLT vaccine formulation provided 100% protection from multiple high-dose FEN administrations whereas 33% of un-vaccinated rats showed untimely demise.

7) The biochemical and behavioral effects described in this application were produced following administration of a small concentration of conjugate and adjuvant. Low doses of vaccine that elicit high antibody levels may be associated with a lack of adverse events in humans.

8) The effect of preventing fentanyl from entering the central nervous system in vaccinated rats was highly robust and enduring and a key factor in the vaccine's therapeutic potential for treating opioid use disorder and preventing fentanyl-induced overdose deaths.

1. An adjuvanted conjugate opioid vaccine, comprising:
a protein-hapten conjugate, wherein the protein-hapten conjugate comprises a protein carrier, wherein the protein carrier is CRM197, and at least one opioid backbone hapten conjugated to the protein carrier, wherein the opioid backbone hapten is fentanyl; and
at least one adjuvant admixed with the protein-hapten conjugate, wherein the at least one adjuvant is dmLT, LTA1, or a combination thereof.

2. (canceled)

3. The adjuvanted conjugate opioid vaccine of claim 1, wherein the at least one opioid backbone hapten is conjugated to the protein carrier through a linker.

4. The adjuvanted conjugate opioid vaccine of claim 1, wherein and the fentanyl is conjugated to the CRM197 through a glutaryl linker.

5. The adjuvanted conjugate opioid vaccine of claim 1, wherein the protein-hapten conjugate comprises between 1 and 15 opioid backbone haptens.

6. (canceled)

7. The adjuvanted conjugate opioid vaccine of claim 1, wherein the protein-hapten conjugate has a structure of: 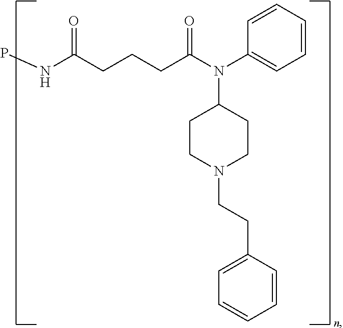
wherein P is a protein carrier and n is between 1 and 15.

8. A pharmaceutical composition comprising a therapeutically effective amount of the adjuvanted conjugate opioid vaccine of claim 1 and a pharmaceutically acceptable excipient, carrier, buffer, stabilizer, or mixture thereof.

9. A method of preventing or treating opioid use disorder or opioid overdose in a subject comprising administering the pharmaceutical composition of claim 8 to the subject.

10. A method of inhibiting effects of opioids on a subject comprising administering the pharmaceutical composition of claim 8 to the subject.

11. A method of inhibiting effects of opioids on a subject, comprising:
administering a pharmaceutical composition to the subject, wherein the pharmaceutical composition comprises a therapeutically effective amount of an adjuvanted conjugate opioid vaccine and a pharmaceutically acceptable excipient, carrier, buffer, stabilizer, or mixture thereof, wherein the adjuvanted conjugate opioid vaccine comprises a protein-hapten conjugate and at least one adjuvant admixed with the protein-hapten conjugate, wherein the protein-hapten conjugate has a structure of: 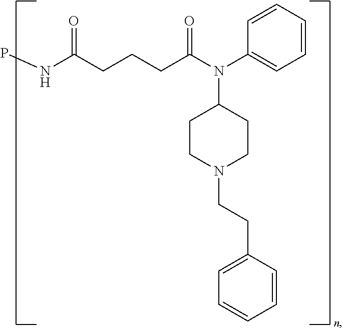
wherein P is CRM197 and n is between 1 and 15, and wherein the at least one adjuvant is dmLT, LTA1, or a combination thereof.

12. The method of claim 11, wherein the at least one adjuvant is dmLT and the pharmaceutical composition is administered to the subject through mucosal routes.

13. The method of claim 11, wherein the at least one adjuvant is LTA1 and the pharmaceutical composition is administered to the subject through intranasal routes.
